(19) United States
(12) Patent Application Publication
(10) Pub. No.: US 2026/0057992 A1
(43) Pub. Date: Feb. 26, 2026

(54) SYSTEM FOR PROCESSING PHARMACEUTICAL PACKAGING MEANS, METHOD FOR OPERATING SUCH A SYSTEM, AND MONITORING DEVICE

(71) Applicant: Bausch + Stroebel SE + Co. KG, Ilshofen (DE)

(72) Inventors: Nico Butzer, Ilshofen (DE); Oliver Lang, Ilshofen (DE); Philip Messerschmidt, Ilshofen (DE); Benjamin Spang, Ilshofen (DE)

(21) Appl. No.: 19/373,067

(22) Filed: Oct. 29, 2025

Related U.S. Application Data
(63) Continuation of application No. PCT/EP2024/060771, filed on Apr. 19, 2024.

(30) Foreign Application Priority Data
Apr. 30, 2023 (DE) 10 2023 001 784.1
May 2, 2023 (DE) 10 2023 111 289.9

(51) Int. Cl. G16H 20/13; G16H 15/00; G16H 40/67

(52) U.S. Cl. CPC G16H 20/13; G16H 15/00; G16H 40/67

(57) ABSTRACT

A system for processing pharmaceutical packaging includes an apparatus with a processing chamber and processing station. An insertion opening is formed in a wall of the apparatus. A transportable container includes an interior for receiving a pharmaceutical production element and a container opening that can be closed and opened via a closing element. The container is dockable to the apparatus. The production element is transferable from the interior into the processing chamber when the closing elements are open. A monitoring device, which includes at least one reading unit and at least one identification element, is arranged on or part of the production element. State information about the production element is stored on or in the identification element, for example in a memory member of the identification element. The state information is readable contactlessly by the reading unit. The system can be monitored by a monitoring method.

### CROSS-REFERENCE TO RELATED APPLICATIONS

This application is a continuation patent application of international application number PCT/EP2024/060771, filed on Apr. 19, 2024, and claims the benefit of German application number 10 2023 001 784.1, filed Apr. 30, 2023 and of German application number 10 2023 111 289.9, filed May 2, 2023, which are incorporated herein by reference in their entireties and for all purposes.

### FIELD

The present disclosure relates to a system for processing pharmaceutical packaging means, which comprises an apparatus for processing the packaging means, wherein the apparatus comprises a processing chamber and at least one processing station, which is arranged in the processing chamber, for the packaging means, and wherein an insertion opening that is capable of being closed and opened via a closing element is formed in a wall of the apparatus.

In addition, the present disclosure relates to a monitoring device for such a system and to a method for operating a system for processing pharmaceutical packaging means.

### BACKGROUND

In the apparatus of the system of the type mentioned at the outset, pharmaceutical packaging means can be processed at the at least one processing station. The packaging means may be primary packaging means, such as in particular containers that can be filled with a pharmaceutical product. The containers may, for example, be vials, syringes, cartridges, or ampoules. The containers may be stable or non-stable. Alternatively or additionally, the packaging means may, for example, be secondary packaging means, which in turn receive primary packaging means, for example tubs, trays, or nests. The at least one processing station may, for example, be an unpacking station for the secondary packaging means, a weighing station, a filling station for filling the primary packaging means, or a closing station for closing them. Other types of processing stations are conceivable.

It is known that packaging means and pharmaceutical production elements can be fed through the insertion opening. For this purpose, there are transportable containers that can be docked to the apparatus. With the container and the insertion opening open, the packaging means and the production elements are removed from the container and transferred to the processing chamber for processing.

In the present example, the packaging means can be regarded as pharmaceutical production elements, which can also be referred to as pharmaceutical production materials. Other types of production elements are, for example, machine parts, in particular of the at least one processing station, or format parts, which are adapted, for example, to the packaging means to be processed.

It is desirable to be able to ensure the highest possible process reliability during operation of the apparatus, wherein the production cycle can preferably be traced as comprehensively as possible. For process reliability, it may be advantageous to instruct a user and to avoid any incorrect operation of the apparatus or of the system.

An object of the present disclosure is to provide a system for processing pharmaceutical packaging means with an apparatus for processing the packaging means, in which increased process reliability can be ensured.

### SUMMARY

In a first aspect of the present disclosure, a system for processing pharmaceutical packaging means is provided. The system comprises:
an apparatus for processing the packaging means. The apparatus comprises a processing chamber and at least one processing station for the packaging means and being arranged in the processing chamber, wherein an insertion opening that is capable of being closed and opened via a closing element is formed in a wall of the apparatus,
a transportable container, which comprises an interior for receiving at least one pharmaceutical production element and a container opening that is capable of being closed and opened via a closing element. The container is dockable to the apparatus, wherein the at least one production element comprises a production element, which is transferable from the interior into the processing chamber when the closing elements are open, and
a monitoring device, which comprises at least one reading unit and at least one identification element. The identification element is arranged on or comprised by the at least one production element, wherein state information about the production element is stored on or in the at least one identification element. In particular, the state information is stored in a memory member of the identification element, wherein the state information is readable contactlessly by means of the at least one reading unit.

In a second aspect of the present disclosure, a method in accordance with the first aspect for monitoring a system of the type of the first aspect provides that state information of at least one identification element is read by means of the at least one reading unit.

### BRIEF DESCRIPTION OF THE DRAWINGS

The foregoing summary and the following description may be better understood in conjunction with the drawing figures, of which:

FIG. 1: shows a schematic representation of the system in accordance with the present disclosure in a preferred embodiment;

FIG. 2: shows a schematic representation of a production element of the system, comprising an identification element, and of a reading unit;

FIG. 3: shows a schematic representation of an apparatus for processing pharmaceutical packaging means as part of the system of FIG. 1; and

FIG. 4: shows a schematic representation of a preparation device and a supply device of the system of FIG. 1.

### DETAILED DESCRIPTION

Although the present disclosure is illustrated and described herein with reference to specific embodiments, the present disclosure is not intended to be limited to the details shown. Rather, various modifications may be made in the details within the scope and range of equivalents and without departing from the present disclosure.

The present disclosure relates to a system for processing pharmaceutical packaging means. The system comprises:
an apparatus for processing the packaging means. The apparatus comprises a processing chamber and at least one processing station for the packaging means. The processing station is arranged in the processing chamber. An insertion opening that is capable of being closed and opened via a closing element is formed in a wall of the apparatus.
a transportable container, which comprises an interior for receiving at least one pharmaceutical production element and a container opening that is capable of being closed and opened via a closing element. The container is dockable to the apparatus, wherein the at least one production element comprises a production element, which is transferable from the interior into the processing chamber when the closing elements are open.
a monitoring device, which comprises at least one reading unit and at least one identification element. The identification element is arranged on or comprised by the at least one production element, wherein state information about the production element is stored on or in the at least one identification element. In particular, the state information is stored in a memory member of the identification element, wherein the state information is readable contactlessly by means of the at least one reading unit.

Production elements that are used in the processing of pharmaceutical packaging means are provided in the system in accordance with the present disclosure. At least some of the production elements can be received in the transportable container. The container can be docked to the apparatus and, in particular, be detachably connected to the apparatus. In particular, the container is docked to the outside of the apparatus, for example to a wall of the apparatus. Once the container is docked to the apparatus and the corresponding closing element is opened, a production element received in the container can be transferred into the processing chamber and used there. In accordance with the present disclosure, the system comprises a monitoring device, wherein an identification element is arranged on or comprised by at least one production element. State information can be read contactlessly via at least one reading unit. The state information advantageously provides the possibility to check and/or trace the use of production elements. The possibility to monitor and/or track production elements over time during the production cycle is preferably provided. For this purpose, the state information comprises, for example, identification numbers of the production elements and preferably further information that characterizes their properties in detail. This is discussed below. Read state information can be processed by the system and preferably checked for compliance with an optional production plan. The production plan may, for example, store which production elements are required for a planned production of containers and, optionally, which state the production elements must assume or have. This information may be time-dependent. Checking the state information preferably makes it possible to monitor compliance with the production plan, to intercept or preferably avoid any disruptions during execution and/or to prepare production elements for production, which in turn can ensure higher process reliability.

In the present case, “contactless” does not exclude that the at least one reading unit is in physical contact with the production element when reading the state information.

It is understood that the system may comprise more than one apparatus. The apparatuses may be configured identically or differently.

In a corresponding manner, the system may comprise more than one container. The containers may be configured identically or differently.

Advantageously, the system comprises at least one data processing device with a memory unit, in which state information of the at least one production element is stored. The state information can be managed and/or modified and preferably processed via the data processing device. The data processing device preferably makes it possible to create and/or modify state information, which can be written into the memory member of the identification element via the at least one reading unit. This is discussed below.

The at least one data processing device can be part of a production system (MES, Manufacturing Execution System).

Redundant storage of state data in the identification elements and in at least one data processing direction may be provided.

The state information is preferably linked to a production plan relating to the processing of the packaging means and/or the planned use of the at least one production element in the apparatus. The system preferably provides the possibility to create a production plan, which stores which production elements are used in what way and/or which packaging means are processed how. By linking the production plan with the state information, correct execution can preferably be prepared, monitored, and/or ensured. In particular, it is provided that each production element is used in accordance with the production plan and, if necessary, also assumes a correct state (for example, by checking that the production element is in a cleaned or autoclaved state, by checking whether it has already been used or not, etc.).

The production plan preferably also provides the possibility of planning and/or monitoring partial or complete autonomous operation of the system by storing information about the production elements to be used in the production plan, wherein the system and in particular the apparatus can be controlled on the basis of the state information.

The at least one data processing device can be positioned locally on the system and, for example, on the apparatus. It is also conceivable that the at least one data processing device is positioned spatially remote from the apparatus and is formed by one or more servers, for example in a cloud.

Changes to the state information of the at least one production element can preferably be stored in the memory unit. This makes it possible to update specific state information and, for example, to create a log about the use of the production element. Preferably, there is the possibility to track the production element seamlessly, for example over the entire production cycle or even life cycle. This may, for example, extend from the supplier via internal logistics, production to delivery and disposal of the production element.

The at least one data processing device can preferably create a report on the use of the at least one production element on the basis of the state information.

It is advantageous if the at least one data processing device is configured to create a report on the execution of the production plan, which allows production processes to be monitored.

For example, depending on the use and/or consumption of the at least one production element, procurement information for procuring further production elements is transmittable from the at least one data processing device to a spatially remote delivery device. For example, changes in the stock of the production element are registered and a preferably automatic ordering process is initiated, which allows for efficient production planning and warehouse management.

It may also be advantageous if, through the processing of the state information by the at least one data processing device, warehouse management with regard to storage locations of production elements, costs for warehousing, routes and/or positions of production elements become transparent and are preferably seamlessly traceable. Automatic picking of production elements on the basis of the evaluated state information is conceivable.

It may be provided that the at least one reading unit forms the at least one data processing device, or vice versa.

Alternatively, the at least one data processing device is formed separately from the at least one reading unit and coupled thereto.

Information can preferably be transmitted wirelessly between the at least one data processing device and the at least one reading unit.

It is advantageous if the state information of the at least one identification element is modifiable, in particular by means of at least one reading unit, for example with a respective reading unit. This allows a particular state of the identification element to be adapted when the state of the production element changes. For example, it is conceivable to mark the production element as cleaned, used, or the like. The reading unit(s) can form a writing instrument or writing instruments according to the above.

In addition, it is preferably possible to create the state information in the identification element, for example to write state information to the memory member for the first time. This can be carried out, for example, during production and/or before the planned use of the production element.

The memory member of the at least one identification element may comprise two or more memory portions, which differ from one another in terms of their nature. In particular, a memory portion whose content is only readable and/or a memory portion whose content is readable and modifiable or which is writable is provided. In practice, it may be advantageous if part of the state information is static and cannot be modified. For example, this memory portion stores information that cannot or should not change during the life cycle of the production element. On the other hand, a memory portion which stores information that can change may be provided so that it is possible to update the state information.

It may be provided that a memory portion that can be read only by using a specific reading unit is present.

Alternatively or additionally, a memory portion that can be read with any of the reading units may be provided.

It may be provided that a memory portion in which state information can be written with only one specific reading unit or in which state information can be modified with the specific reading unit is present.

Furthermore, it may be provided that a memory portion in which state information can be modified with any reading unit or to which state information can be written with any reading unit is present.

The above configuration allows, for example, different types of state information to be stored in the memory member on the manufacturer side on the one hand and on the user or customer side on the other hand. For example, manufacturer-side state information is used to control the apparatus, and customer-side state information can relate to the production cycle, for example.

The at least one production element may, for example, be a format part of the apparatus, wherein different types of format parts differ from one another by at least one packaging-means-specific property.

The at least one production element may, for example, be a machine part, for example the at least one processing station.

The at least one production element may, for example, be a pharmaceutical packaging means.

The at least one production element may, for example, be a filling element, which is used when filling the packaging means with a product. Possible filling elements include, for example, a product container for the product, a fluid conduit through which the product is conveyed, or a filling needle.

It has already been mentioned that production elements that are inserted into the processing chamber may be provided. It is conceivable that production elements that remain outside the processing chamber are provided. Such production elements may advantageously also have an identification element.

The at least one production element is, for example, a receiving body for temporarily receiving a different type of production element, in particular in the interior of the container. The receiving body is arranged, for example, in the container and serves to temporarily receive a production element to be introduced into the processing chamber. The receiving body may, for example, be a magazine plate for production elements, which is arranged in the container.

The receiving body may be arranged in a pull-out manner in the container in order to be able to load it more easily or to remove production elements more easily.

The receiving body may, for example, be removed from the container or pulled out manually and/or with the, in particular, robotic handling device.

As mentioned above, at least one pharmaceutical production element can be received in the interior. The system may, for example, comprise production elements that are used during production and that are arranged or remain outside the apparatus and therefore are not or do not need to be arranged in the interior of the container.

In particular, it may be provided that the container itself is a pharmaceutical production element and comprises an identification element that can be read by the at least one reading unit. The container is also preferably monitored. State information relating to the container may, in addition to an identification number, be information relating to the current content of the container. For example, it is stored which production elements are received in the container and in which position they are each located.

In a preferred embodiment of the present disclosure, the monitoring device is formed to be radio-based for reading the at least one identification element by means of electromagnetic radiation, in particular by means of RFID. Accordingly, the at least one reading unit can be an RFID reader. The identification element can be an RFID tag or RFID chip. It is also preferably possible for data to be written to or modified in the memory member of the identification element by means of the reading unit, which can thus be an RFID writing instrument.

Alternatively, it may be provided that the monitoring device is configured as an optical scanning device for detecting the at least one identification element by means of light. For example, a code (barcode or matrix code) readable via the scanning device is used.

In particular when using RFID technology, it is advantageous if the at least one identification element is arranged in the at least one production element and, in particular, is free of contact with the outer surface thereof and/or is encapsulated in the production element.

In the last-mentioned advantageous embodiment, it is preferably possible to sterilize and/or autoclave the at least one production element with the at least one identification element without destroying the latter. For the latter purpose, the production element is preferably heat-resistant.

Preferably, the production element can be subjected to an H₂O₂ decontamination cycle without destroying the identification element.

The production element, including the identification element, is preferably resistant to cleaning agents.

Possible materials for the at least one production element include, for example, a sterilizable and/or autoclavable plastic such as PEEK or a pharmaceutical-grade stainless steel, e.g., of the type AISI 316L.

The system preferably comprises at least one handling device in the processing chamber, wherein the at least one production element and/or the packaging means are capable of being handled by means of the at least one handling device. The at least one handling device is preferably configured to be robotic.

The system may comprise a supply device for storing a plurality of containers, wherein the supply device is arranged outside the apparatus. For example, for later use, containers are prepared with the preparation device mentioned below and are stored in the supply device, which is configured as or comprises a shelf for this purpose. The memory unit of the at least one data processing device preferably stores a position of each container in the supply device in order to be able to identify them at a later point in time.

The system may comprise a preparation device which is spatially separated from the apparatus and comprises a working space, wherein a removal opening that is capable of being closed and opened via a closing element is formed in a wall of the preparation device, wherein the container is dockable to the preparation device, and wherein the at least one production element is transferable from the working space into the interior when the closing elements are open. The preparation device can be used to load the at least one container with production elements. For this purpose, the at least one production element is transferred through the openings into the docked container. The container can be docked to the preparation device and, in particular, can be detachably connected to the preparation device. The container is docked in particular to the outside of the preparation device, for example to a wall of the preparation device.

For later use, the container is finally closed and undocked and may, for example, be stored in the optional supply device in the meantime.

Advantageously, it is provided that state information of the container is updated during equipping via the production device. For example, the memory member stores the type of production element received in the interior and the position at which it is located. It is conceivable, for example, that a receiving body (for example, a magazine plate) is arranged in the interior, wherein the identification element of the container stores the position at which the receiving body is located and, if applicable, where a corresponding production element is located on the receiving body. The receiving body may, for example, have an independent identification element, which stores the position of a production element on the receiving body.

When equipping the container via the preparation device, it is preferably possible to ensure via at least one reading unit that the container is equipped correctly, in particular in accordance with a production plan. For example, the state information of corresponding production elements must be read, which are inserted into the container only if they are the correct production elements and if they have the correct state (for example, cleaned, sterilized, or unused). In this way, a high level of process reliability can be ensured and the production cycle can be reliably traced. Any indications for production elements to be introduced into the container and/or relating to the container may be provided, for example, on an indicator unit of the reading unit or on a separate indicator unit, for example on a display. In this way, the user can be instructed to equip the container, for example in a step-by-step instruction.

It may be advantageous if indications have to be acknowledged by the user, for example step by step. Acknowledgment can be carried out via the indicator unit or via a separate input unit. For example, state information, such as that of the identification element of the container, is written to or updated in the memory member only if all actions have been acknowledged by the user. This increases process reliability and ensures tracing of the individual actions.

It may be provided for the system to comprise more than one preparation device. The preparation devices may be configured identically or differently.

The closing elements of the apparatus and/or of the preparation device on the one hand and of the container on the other hand are preferably standardized. In particular, a standardized port is formed, in particular an RTP (Rapid Transfer Port). The container may be a so-called beta container with a beta port, wherein the corresponding alpha port is arranged on the apparatus and/or on the preparation device.

The system may preferably comprise a plurality of reading units, which are arranged at different positions on the apparatus. The reading units may be configured identically or differently.

The at least one reading unit may be stationary. Alternatively or additionally, at least one movable, in particular user-guided, reading unit is provided.

For example, a reading unit that is user-guided may be arranged outside the apparatus.

For example, a reading unit that is user-guided may be arranged in the processing chamber.

A reading unit may be arranged on or fixed to the at least one processing station.

For example, a reading unit may be arranged on or fixed to the at least one handling device in the processing chamber. For example, the reading unit is arranged on a tool or end effector of the, for example robotic, handling device. When the container is docked, identification elements of production elements in the interior of the container may, for example, be read via this reading unit. For example, it is provided that a production element is inserted into the processing chamber only if it is the correct production element, in particular in accordance with the production plan, and specifically has a specified state.

For example, a reading unit is arranged or fixed at the insertion opening, preferably on/to the outside of the apparatus. This reading unit can advantageously be used to read the identification element of a container that is docked to the apparatus or that is to be docked to the apparatus. This can ensure, preferably before the closing element is opened, that the correct container with the correct content is being docked or is docked. This ensures a high level of process reliability and avoids errors in the production cycle.

A reading unit may, for example, be arranged on or fixed to a receiving element in the processing chamber for receiving a used or discarded production element.

If the preparation device is provided, a reading unit, which is preferably user-guided, may be arranged in the working space, for example.

A reading unit may be arranged on or fixed to an, in particular, robotic handling device in the working space of the preparation device.

A reading unit may be arranged or fixed at the removal opening, preferably on/to the outside of the preparation device, wherein the identification element of a container that is docked to the preparation device or that is to be docked to the preparation device is readable via the reading unit. In the same way as with the apparatus, this ensures that the correct container is loaded with production elements. Preferably, the closing elements are opened only if it is ensured that the correct container is docked and has the specified state.

It is understood that an exchange of information via the at least one data processing device may be possible for the plurality of reading units.

The state information may, for example, comprise or relate to at least one of the following:
An identification number of the production element, which is in particular unique and on the basis of which the production element can preferably be unambiguously identified.
A part number of the production element. This could be an article number, for example.
A type of the production element.
A batch of the production element.
Information relating to the content of the production element in the case of a container as a production element.
Information relating to the position of a further production element in the case of a container that receives the further production element.
Information relating to the position of a further production element in the case of a receiving body that receives the further production element.
Information relating to the geometry of the production element. This information may, for example, be taken into account by the handling device when handling the production element in the processing chamber.
Information relating to the installation of the production element in the apparatus, in particular a target position in the apparatus. For example, the handling device is controlled depending on the information for installing the production element.
Information relating to a control of the apparatus using the production element in the apparatus.
Information relating to a processing of the packaging means by the apparatus using the production element in the apparatus.
Information indicating whether the production element has been used or has not yet been used. This makes it possible to increase process reliability. For example, only unused production elements are used and are marked as used after their use.
Information indicating whether the production element, in the case of a container, has been previously opened or not. This allows the process reliability to be increased. For example, it can be ensured that only unopened containers are docked to the apparatus. If it is assumed that the unopened container has the correct level of cleanliness, it is possible, for example, to avoid the risk of compromising the cleanliness of the processing chamber.
Information indicating whether the production element is contaminated or not, for example when used with a toxic product;
Information as to whether the production element has been cleaned or sterilized or not. This also allows the process reliability to be increased. For example, the container is docked or is opened for transferring the production element into the processing chamber only if it is ensured that the production element received in the container is sterilized. The state information in this regard is preferably stored in the identification element of the container itself.
A timestamp, for example for the point in time of creating and/or the point of time of changing the state information.

The state information may comprise the identification of an apparatus on or in which the production element is to be used and/or has been used.

The state information may comprise the identification of a preparation device at which the production element was inserted into the container or at which the container was equipped.

The state information may comprise a timestamp of the use of the production item.

The state information may comprise a timestamp for determining a validated standing time in the sterile status.

On the basis of the state information of the identification element of the at least one container, at least one of the following can be checked during or before docking:
Is the container the right type?
Does it contain the correct content (for example, the type of receiving body and its content)?
At which positions are the required production elements located?
Is the container allowed to be opened or has it already been opened at another apparatus or in another process?
Does the container have a free storage location for reserving a receiving body?
What state (contaminated/cleaned/sterile) does the container have?

The state data regarding the content may, for example, be updated during each loading and/or unloading. The state data are thus up-to-date, even if the container is loaded and/or unloaded over multiple docking cycles. For increasing process reliability, the closing elements are preferably opened only if all state data are correct or plausible.

The identification element of receiving bodies can store their state information. At least a portion of the state information can be written into the identification element of the container during insertion into the container. As a result, the content of the container can also be read from the outside. Possible state information of the identification element of the receiving body may comprise:
Number of storage locations and/or identification of which storage location is free or occupied;
Positions of the received production elements;
Which production element is it?
Dimensions and/or type of the production element;
What state (contaminated/cleaned/sterile) does the receiving body have?

The state information on the content may, for example, be updated during each loading and/or unloading.

The state information of the identification element of a format part may, for example, comprise:
What type of format part is it?
What properties does the format part of this type have?
Insertion position and removal position for the handling apparatus?
Dimensions of the format part, for example height and/or diameter;
Designation of the format part in various languages;
What state (contaminated/cleaned/sterile) does the format part have?

In order to avoid accidental or intentional misuse of data, it may be provided that corresponding access rights are required. For example, there may be password-protected regions and user profiles with different access rights in order to access the at least one data processing device and/or the identification elements.

The process reliability may, for example, be increased by reading check information (e.g., a checksum or the like) as part of the state information.

The apparatus may comprise a control device, which is directly or indirectly coupled to the at least one reading unit, wherein the apparatus is controllable depending on the determined state information.

The data processing device may comprise or form the control device, or the data processing device may be formed separately from the control device and be coupled thereto.

Depending on the determined state information, the control device can, for example, control at least one processing station and/or at least one handling device, which is arranged in the processing chamber, for the production element and/or the packaging means.

Alternatively or additionally, depending on the determined state information, the control device can, for example, control at least one indicator unit for the user to provide at least one indication. The at least one indicator unit preferably comprises a controllable image display.

The at least one reading unit may comprise or form the at least one indicator unit. Alternatively, the indicator unit may be spatially separated from the reading unit.

For example, a confirmation can be provided on the at least one indicator unit in the event that a production element identified on the basis of the state information is a correct production element, in accordance with the production plan, for example when equipping the container via the preparation device.

For example, an error indication can be provided on the at least one indicator unit in the event that a container that does not correspond to the production plan is docked or intended to be docked to the apparatus.

For example, an error indication can be provided on the at least one indicator unit in the event that a container that assumes a state that deviates from the production plan because, for example, the container has been opened and/or is not sterilized or does not contain a non-sterilized production element is docked or intended to be docked to the apparatus.

For example, an error indication can be provided on the at least one indicator unit in the event that a container that does not contain at least one production element specified in the production plan or that does not contain it in a specified state (because, for example, it is not sterilized or has already been used) is docked or intended to be docked to the apparatus.

For example, an error indication can be provided on the at least one indicator unit in the event that the container is docked or intended to be docked to the apparatus at an insertion opening deviating from an insertion opening specified in the production plan. For example, the apparatus has multiple insertion openings with respective closing elements, wherein the container is to be docked at a specific insertion opening. If docking has occurred or is planned at the wrong insertion opening, an error indication may be issued.

For example, a confirmation can be provided on the at least one indicator unit when a container that is in accordance with the production plan is docked or intended to be docked to the apparatus. If all requirements are met, this is preferably acknowledged.

It can be provided that the system comprises at least one self-propelled and self-steering transport device for receiving at least one container, wherein the container is capable of being autonomously docked to and decoupled from the apparatus via the transport device. As a result, the system has a high versatility. Via the transport device, a container can, for example, be transported from the preparation device to the supply device and, when in use, from there to the apparatus.

The at least one transport device preferably comprises at least one reading unit, or such a unit is arranged on the transport device. This allows, for example, identification elements of containers to be read.

As mentioned at the outset, the present disclosure also relates to a monitoring device. A monitoring device in accordance with the present disclosure is a monitoring device for a system for processing pharmaceutical packaging means of the type described above.

The monitoring device comprises in particular at least one reading unit and at least one identification element. Advantageously, the at least one data processing device is part of the monitoring device. The control device may be part of the monitoring device.

Advantageous embodiments of the monitoring device in accordance with the present disclosure result from the advantageous embodiments of the system in accordance with the present disclosure, as described above.

As mentioned at the outset, the present disclosure further relates to a method. A method in accordance with the present disclosure for monitoring a system of the type described above provides that state information of at least one identification element is read by means of the at least one reading unit.

The advantages explained in connection with the system in accordance with the present disclosure can also be achieved using the method. Advantageous exemplary embodiments of the method follow from advantageous embodiments of the system in accordance with the present disclosure. Reference is made to the above statements.

In particular, the method can provide for the apparatus to be controlled depending on the state information. When placing an identification element in the container, the identification element of said container can be written to. When the identification element is removed from the container, the identification element of said container can be updated by changing the state information. Production elements can be marked as used or unused, as cleaned or not cleaned, as sterile or non-sterile, etc. Reference is made to the above statements.

The following description of preferred embodiments of the present disclosure serves in conjunction with the drawing to explain the present disclosure in more detail. In the figures:

The present disclosure is described below using the example of a preferred embodiment of the system in accordance with the present disclosure, which is denoted overall by reference numeral 100. Reference is first made at this point to the above explanations, which in particular describe preferred embodiments of the system and from which advantageous exemplary embodiments of the method follow. Unless explained below, the above-described optional features of the system may be present and implemented in whole or in part in the embodiment presented here.

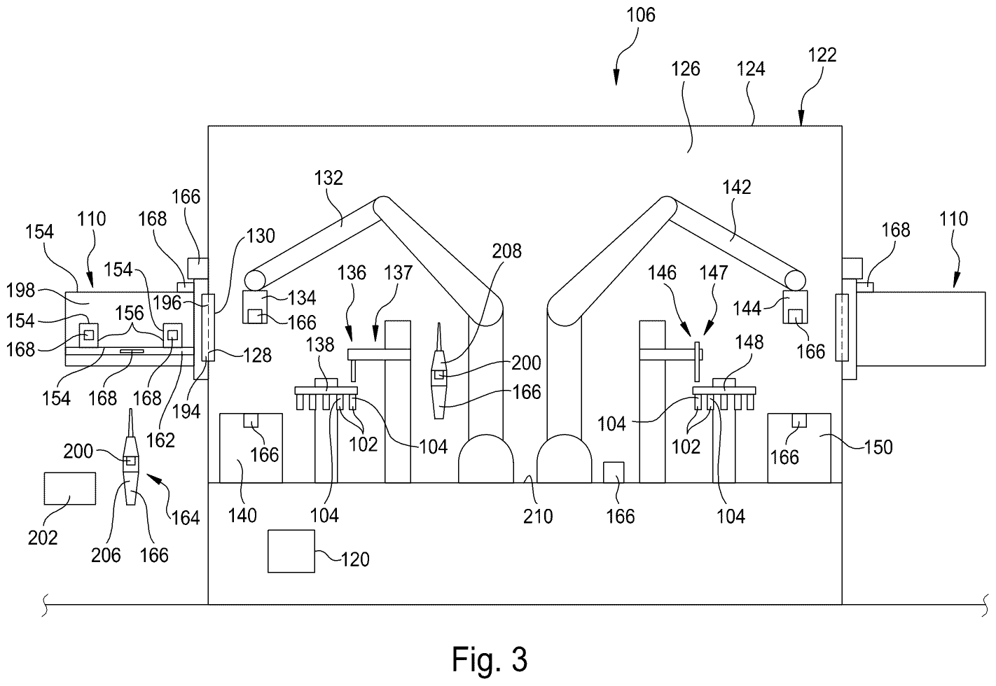

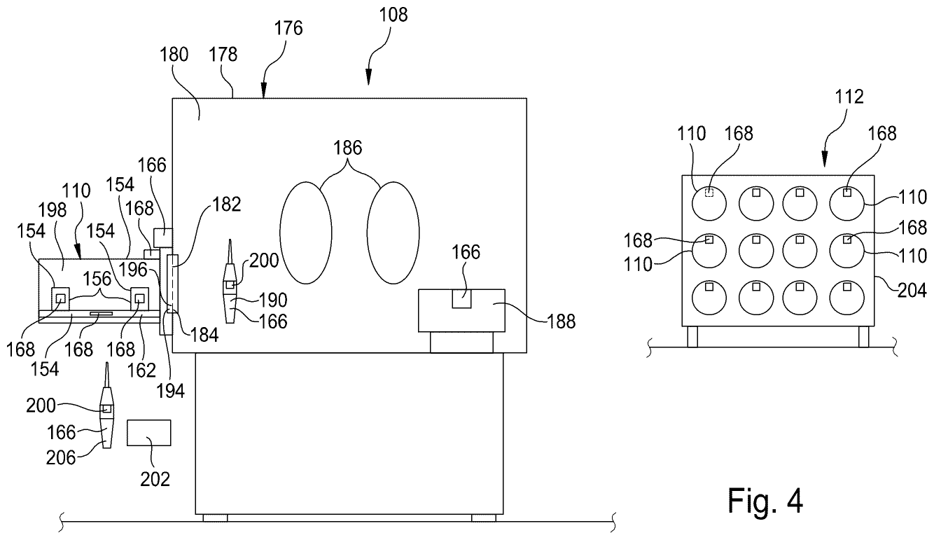

The system 100 is used to process pharmaceutical packaging means 102. By way of example, FIG. 3 represents containers 104 as primary packaging means 102, wherein the containers are, for example, vials. Other types of containers 104 are conceivable.

A pharmaceutical product is filled into the containers 104 by means of an apparatus 106 for processing the packaging means 102. The containers 104 are subsequently closed. The apparatus 106 is part of the system 100.

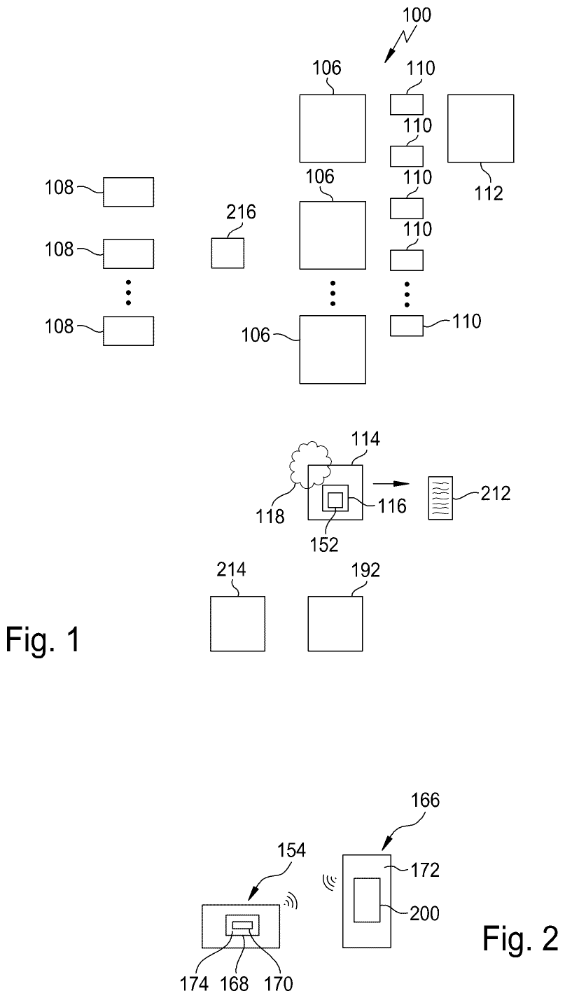

As can be seen in particular in FIG. 1, the system 100 in the present example comprises, but this is not limiting, a plurality of apparatuses 106, which may be configured to be identical or of different types.

In addition, the system 100 advantageously comprises at least one preparation device 108, of which multiple preparation devices 108 are provided in the present example.

The system 100 comprises at least one transportable container 110, wherein multiple containers 110 are also provided in the present case.

Advantageously, at least one supply device 112 is provided for the containers 110.

Furthermore, the system 100 in the present case comprises a data processing device 114, which comprises a memory unit 116. In the present case, the data processing device 114 is formed spatially separated from the apparatuses 106 and may, for example, comprise one or more servers. It is conceivable that this can be formed by a schematically represented cloud 118.

Each apparatus 106 comprises a control device 120, wherein said control device may be part of the data processing device 114 or may be formed by the data processing device 114. In this example, the control device 120 is coupled to the data processing device 114 for data exchange.

At the system 100, the packaging means 102 are processed in the apparatus 106. The apparatus 106 comprises a chamber 122, which has a wall 124 and a processing chamber 126 enclosed by this wall. At least one insertion opening 128, which can be closed via a closing element 130 arranged thereon or can be opened when the closing element 130 is open, is formed in the wall 124. In the present case, two insertion openings 128 and closing elements 130 are shown schematically in FIG. 3.

A first handling device 132 is arranged in the processing chamber 126. The handling device 132 in the present case is configured to be robotic, for example by means of a multi-axis robot. The handling device 132 has a tool 134 for handling production elements and packaging means 102.

A first processing station 136 is a filling station 137, at which the containers 104 can be filled with the pharmaceutical product. For this purpose, the containers 104 can be held in a holder 138. A receiving element 140 can be used to temporarily set down the containers 104.

In the present case, the apparatus 106 comprises a second handling device 142, which can preferably be configured to be identical to the handling device 132 and in particular can be configured to be robotic. The handling device 142 comprises a tool 144.

A closing station 147, with which the containers 104 can be closed after filling, is provided as a second processing station 146. For this purpose, the containers 104 can be held on a holder 148. A further receiving element 150 can be used to temporarily set down the containers 104.

When using the apparatus 106, the packaging means 102 and a container for the product are introduced into the processing chamber 126, for example via the insertion opening 128. The containers 104 are filled, subsequently closed, and again removed from the processing chamber 126 via an opening, for example the insertion opening 128.

For controlling and managing the processing of packaging means 102, a production plan 152 is stored in the memory unit 116, for example. The production plan 152 stores, for example, which packaging means 102 are filled with which product, in which apparatus 106 this takes place, and which production elements 154 are to be used.

Production elements 154 can be the packaging means 102 themselves. Further production elements are, for example, format parts 156, which are used in the apparatus 106 and have container-specific properties.

Furthermore, machine parts, for example, can be provided as production elements 154, for example as part of the handling device 132, 142.

In the present example, the containers 110 are provided as further production elements 154, which remain outside the apparatus 106 but are used during production and can in turn receive other types of production elements 154, such as the format parts 156.

In the present example, further production elements 154 are receiving bodies 158, in particular in the form of magazine plates, which are arranged in a pull-out manner in the container 110 and in turn serve to temporarily receive further production elements 154, for example the format parts 156.

The production element 154 may, for example, be a filling element that is used when filling the containers 104 with the product to be filled. These include, for example, a container for the product, a fluid conduit or a filling needle for filling the product into the containers 104.

By way of example, reference numeral 156 in the drawing denotes two format parts, which are used as an example for explaining the present disclosure in more detail below. Reference numeral 162 denotes a magazine plate.

In order to monitor the production cycle and increase process reliability, in particular with regard to compliance with the production plan 152, the system 100 comprises the monitoring device 164 in accordance with the present disclosure in a preferred embodiment. In the present example, the monitoring device 164 comprises a plurality of reading units 166 as well as identification elements 168, which are comprised by the corresponding production elements 154.

The reading units 166 allow state information stored in a memory member 170 of the identification element 168 to be read contactlessly. In the present case, the reading units 166 are radio-based by means of electromagnetic radiation. The reading units 166 are in particular RFID readers 172, which in the present example are also configured as RFID writing instruments so that the state information can be read as well as, at least partially, modified. Accordingly, new state information can be written to the particular memory member 170, or existing state information can be modified. The identification elements 168 are RFID tags 174 or RFID chips.

FIG. 2 schematically represents a production element 154 with identification element 168. The identification element 168 is received in the production element 154, for example potted therein or positioned in a closed receptacle of the production element 154, so that it is free of contact with the outer surface of the production element 154.

The production element 154 is in particular sterilizable and/or autoclavable, without destroying the identification element 168. The production element 154 can be subjected to an H₂O₂ decontamination cycle without destroying the identification element 168.

Via the monitoring device 164, the production elements 154 can be tracked and their respective state monitored, preferably throughout the entire production cycle. This allows the process reliability in the system 100 to be significantly increased by using the monitoring device 164. Possible advantages achieved by the present disclosure may comprise, for example:
Process reliability through, in particular, unmanned production and seamless tracking of the production elements 154;
More efficient production, in particular through automated logistics and/or automated transport for necessary production elements 154. For example, the transport of containers 110 with correct production elements 154 in the correct state, in particular sterilized, should be mentioned, wherein the containers 110 are made available to a particular apparatus 106 at the correct time.
Timely identification of any bottlenecks in the warehouse stock of production elements 154.
Tracking preferably over the entire life cycle and the entire supply chain, for example from the supplier via production to delivery and disposal.
Detecting changes in the stock of the production elements 154 and balancing through preferably automatic ordering processes with a view to better warehouse management and production planning.
Monitoring a correct production flow and material flow.
Transparency and traceability with regard to warehouse management for storage locations, warehousing costs, routes and positions of production elements 154.
Automatic picking can be provided.
Digitization of the production.
Creation of reports with a view to preferably complete documentation of the production process.
Identification of production elements 154 and/or contents of containers 110 without a view inside or visual contact.

The preparation device 108, which serves to prepare the production process, comprises a chamber 176, which has a wall 178 and a working space 180 enclosed by this wall. At least one removal opening 182 is formed in the wall 178. The removal opening 182 can be selectively closed or opened by means of a closing element 184.

The preparation device 108 can have a glove port 186 for a user to intervene, two ports being shown in the present case.

Alternatively or additionally, the preparation device 108 may comprise a controllable robotic handling device, which may, for example, have a reading unit 166 on the tool, as is the case in the handling devices 132, 142. Via this reading unit 166, state data of the identification elements 168 of production elements 154 can be read and, if necessary, changed,

For example, a receiving element 188 for production elements 154 can be provided in the working space 180.

In the present case, the monitoring device 164 comprises a user-guided reading unit 190 in the working space 180. A further reading unit 166 can be arranged on the receiving element 188 in order to read state information of the production elements 154 stored thereon.

Advantageously, a reading unit 166 is arranged in a stationary manner on the outside of the preparation device 108, for example in the region of the removal opening 182, in order to read state information of a container docked thereto or of a container 110 to be docked thereto.

Prior to production, required production elements 154 can be retrieved from a storage device 192 of the system 100, for example in accordance with the production plan 152, and introduced into the working space 180 for the preparation of a container 110. For the sake of simplicity, it is assumed that the production elements 154, in the present case in the form of the format parts 156, are already sterilized.

State information is stored in the corresponding memory members 170, which state information in the present example comprises in particular the following: identification number, part number, part type, batch number, information about the geometry, marking as a sterilized production element 154, marking as an unused production element 154, information relating to a target position in the apparatus 106, any information for controlling the handling device 132, 142. Further possible state information has already been explained above; reference is made to these statements.

For preparation, a container 110 is docked to the preparation device 108. In particular, the container 110 is docked to the outside of the wall 178. The identification element 168 of the container 110 is read via the reading unit 166 on the outside at the removal opening 182. In particular, the identification number of said container is detected and it is also determined whether said container has the required cleanliness. A closing element 194, which closes a container opening 196, is opened and so is the closing element 130 for the removal opening 182.

The closing elements 130 and/or 184 on the one hand and 194 on the other hand are preferably standardized. In particular, a standardized port is formed, in particular an RTP (Rapid Transfer Port). The container 110 may accordingly be a so-called beta container with a beta port, wherein the corresponding alpha port is arranged at the openings 128, 182.

Production elements 154 can be inserted into the container 110 when the closing elements 184 and 194 are open and an interior 198 of the container 110 communicates with the working space 180.

The user adds the required format parts 156 to the interior 198 of the container 110 in accordance with the specification in the production plan 152. This is accomplished, for example, in that the format parts 156 can be taken from the receiving element 188, which can be detected by the reading unit 166 thereof. By reading the identification elements 168, e.g., via the hand-held reading unit 190, it can be ensured that the format part 156 is the correct one. A confirmation in this regard can be issued on an indicator unit 200 of the reading unit 190 or on an indicator unit 202 separate therefrom. The indicator units 200, 202 preferably have a controllable image display.

The format part 156 is placed on a specified position of the magazine plate 162. The identification element 168 of the container 110 is written to or updated as to the position at which the magazine plate 162 is arranged in the container 110 and as to the position of the magazine plate 162 at which a particular format part 156 is located.

Once the equipping process is completed, the preparation device 108 and the container 110 are closed. The identification element 168 of the container 110 stores that said container is unopened and sterile. For example, a standing time for sterility can be determined via a timestamp of the change.

The data processing device 114 stores the state information of all identification elements 168 as well as the changes to this state information, which are logged accordingly.

The closed container 110 can be undocked from the preparation device 108.

If the container 110 is not immediately brought to the apparatus 106, it can be temporarily stored in the supply device 112. The supply device 112 comprises, for example, a shelf 110 for a plurality of containers 110.

If the container 110 is used at a later point in time, it can be located, for example, by means of a reading unit 206 arranged externally to the apparatus 106 and to the preparation device 108. For this purpose, the identification elements 168 of the containers 110 can be read in order to identify the appropriate container 110.

The container 110 can be docked for use on the apparatus 106. In particular, the container 110 is docked to the outside of the wall 124. A stationary reading unit 166 is preferably arranged on the outside of the wall 124, for example in the region of the insertion opening 128. This allows the identification element 168 of the container 110 to be read, preferably automatically and/or before the container 110 and the apparatus 106 are opened.

This serves, for example, to ensure that at least one of the following is verified or checked: selection of the correct container 110, arrangement of the container 110 at the correct insertion opening 128, in particular verification of the correct content of the container 110, checking whether the production elements 154 received in the interior 198 have the correct state, for example are sterilized and/or unused.

Production elements 154 can be transferred from the container 110 into the processing chamber 126 when the closing elements 130 and 194 are open and the interior 198 communicates with the processing chamber 126.

Only if all requirements specified in the production plan 152 are met are the container 110 and the apparatus 106 opened by opening the corresponding closing elements 130, 194.

In the event of any errors, deviations from the production plan 152, or disruptions, an indication is preferably issued to the user, for example via an indicator unit 200, 202.

Depending on the read state information of the container 110, the apparatus 106 can be controlled via the control device 120. For example, information as to which format parts 156 can be found at which position on the magazine plate 162 is transmitted to the handling device 132.

By means of a reading unit 166 on the tool 134, the identification elements 168 of the format parts 156 can be read, preferably before insertion into the processing chamber 126. If it is ensured that the format parts 156 are the correct ones, the handling device 132 grips the format parts 156 and guides them to the specified target position in the processing chamber 126. In addition to the correctness of the format part 156, it is in particular checked whether said format part has already been used and/or sterilized.

Any temporary storage of production elements 154 can take place on the receiving elements 140, 150. A reading unit 166 is preferably present on each of them in order to detect the presence of the production elements 154.

In addition, a hand-held reading unit 208, which the user can guide, for example, via a glove port 186, can be present in the processing chamber 126.

The format parts 156, which have been inserted into the processing chamber 126, are preferably marked as used. This may, for example, be carried out by updating the state information via the reading unit 166 of a handling device 132, 142, wherein a different reading unit 166 or 208 could also be used.

After production, the format parts 156 can be removed from the processing chamber 126 and disposed of or fed to treatment for further use. The format parts 156 can be received in the interior 198 of the container 110.

After completion of the process, the container 110 can be undocked from the apparatus 106. In particular, the container 110 is closed beforehand.

The apparatus 106 may comprise a further reading unit 166, for example on the floor 210 of the processing chamber 126, wherein said processing chamber can serve to receive discarded production elements 154 (so-called reject plate). The state information for these production elements 154 can be updated to “discarded” or “sorted out.”

The read state information can be used, in particular, to control the apparatus 106, for example a handling device 132, 142 and/or one of the processing stations 136, 146.

After the production cycle, the data processing device 114 can create a report 212 containing all information essential to the production. For creating the report 212, the stored and logged state information in particular is evaluated. The report 212 can be linked to the batch of the pharmaceutical product produced.

Furthermore, the report 212 may comprise an evaluation of which production elements 154 were used or consumed and how frequently they were used. In connection with the aforementioned procurement of the production elements 154 and preferably automated warehouse management, the advantages of state monitoring and predictive maintenance of the system 100 in particular result therefrom.

It may be advantageous if procurement information is transmitted, preferably automatically, from the data processing device 114 to a spatially remote delivery device 214 in order to replenish the stock of the storage device 192.

It has already been mentioned that different types of memory portions can be present in the memory member 170. It is in particular conceivable that there is a memory portion that can only be read. At least one memory portion whose content can be modified may be provided. It is also conceivable that the reading of state information and/or the changing of state information in the particular memory portion is possible only by using a specific reading unit 166. Furthermore, it may be provided that different types of state information are stored in different memory portions. For example, a memory portion for manufacturer-side state information of the production element 154 and a memory portion for customer-side or user-side state information of the production element 154 are present.

The system 100 may comprise a mobile transport device 216. Multiple transport devices 216 may be provided.

The transport device 216 is preferably configured to be self-propelled and self-steering and is formed for the autonomous transport of containers 110 in particular. It is advantageous if a reading unit 166 is arranged on the transport device 216.

The transport device 216 is preferably coupled to the data processing device 114 and/or a control device 120. If necessary, the transport device 216 is started, in particular for the transport of production elements 154. For example, the production elements 154 are fed to the preparation device 108. In this case, moving the containers 110 is in particular conceivable. Equipped containers 110 can be transported by the transport device 216, for example, to the supply device 112 or directly to the apparatus 106. Temporarily stored containers 110 can be picked up from the supply device 112 and brought to the corresponding apparatus 106.

### LIST OF REFERENCE NUMERALS

100 System
102 Packaging means
104, 110 Container
106 Apparatus
108 Preparation device
112 Supply device
114 Data processing device
116 Memory unit
118 Cloud
120 Control device
122, 176 Chamber
124, 178 Processing chamber
128 Insertion opening
130, 184, 194 Closing element
132, 142 Handling device
134, 144 Tool
136, 146 Processing station
137 Filling station
138, 148 Holder
140, 150, 188 Receiving element
147 Closing station
152 Production plan
154 Production element
156 Format part
158 Receiving body
162 Magazine plate
164 Monitoring device
166, 190, 206, 208 Reading unit
168 Identification element
170 Memory member
172 RFID reader
174 RFID tag
180 Working space
182 Removal opening
186 Glove port
192 Storage device
196 Container opening
198 Interior
200, 202 Indicator unit
204 Shelf
210 Floor
212 Report
214 Delivery device
216 Transport device

1. A system for processing pharmaceutical packaging means, the system comprising:
an apparatus for processing the packaging means, which comprises a processing chamber and at least one processing station, which is arranged in the processing chamber, for the packaging means, wherein an insertion opening that is capable of being closed and opened via a closing element is formed in a wall of the apparatus;
a transportable container, which comprises an interior for receiving at least one pharmaceutical production element and a container opening that is capable of being closed and opened via a closing element, wherein the container is dockable to the apparatus, wherein the at least one production element comprises a production element, which is transferable from the interior into the processing chamber when the closing elements are open, and
a monitoring device, which comprises at least one reading unit and at least one identification element, which is arranged on or comprised by the at least one production element, wherein state information about the production element is stored on or in the at least one identification element, wherein the at least one reading unit is configured to read the state information contactlessly.

2. The system in accordance with claim 1,
wherein the system comprises at least one data processing device with a memory unit, in which state information of the at least one production element is stored, linked to a production plan relating to at least one of a processing of the packaging means and a planned use of the at least one production element in the apparatus.

3. The system in accordance with claim 2,
wherein changes to the state information of the at least one production element are storable in the memory unit.

4. The system in accordance with claim 2,
wherein the at least one data processing device is configured to create a report on the use of the at least one production element on the basis of the state information and/or wherein the at least one data processing device is configured to create a report on an execution of the production plan.

5. The system in accordance with claim 2,
wherein depending on at least one of a use and a consumption of the at least one production element, procurement information for procuring further production elements is transmittable from the at least one data processing device to a spatially remote delivery device.

6. The system in accordance with claim 2,
wherein the at least one reading unit forms the at least one data processing device, or the at least one data processing device is formed separately from the at least one reading unit and coupled thereto.

7. The system in accordance with claim 2,
wherein information is transmittable wirelessly between the at least one data processing device and the at least one reading unit and/or

8. The system in accordance with claim 1,
wherein the state information of the at least one identification element is modifiable by means of the at least one reading unit.

9. The system in accordance with claim 1,
wherein the at least one identification element comprises a memory member in which the state information is stored and which comprises two or more memory portions, the memory portions differing from one another in terms of their nature, wherein a memory portion whose content is only readable and/or a memory portion whose content is readable and modifiable or which is writable is provided.

10. The system in accordance with claim 1,
wherein the at least one production element is or comprises at least one of the following:
a format part of the apparatus, wherein different types of format parts differ from one another by at least one packaging-means-specific property;
a machine part of the at least one processing station;
a receiving body for temporarily receiving a different type of production element, in in the interior of the container;
a pharmaceutical packaging means;
a filling element, which is used when filling the packaging means with a product.

11. The system in accordance with claim 1,
wherein the container is a pharmaceutical production element and comprises an identification element readable by the at least one reading unit.

12. The system in accordance with claim 1,
wherein
the monitoring device is formed to be radio-based for reading the at least one identification element by means of electromagnetic radiation and/or by means of RFID.

13. The system in accordance with claim 1,
wherein the monitoring device is configured as an optical scanning device for detecting the at least one identification element by means of light.

14. The system in accordance with claim 1,
wherein the at least one identification element is arranged in the at least one production element, free of contact with the outer surface of at least one production element and/or encapsulated.

15. The system in accordance with claim 1,
wherein the at least one production element with the at least one identification element is sterilizable and/or autoclavable without destroying the at least one identification element.

16. The system in accordance with claim 1,
wherein the system comprises at least one handling device in the processing chamber, wherein the at least one production element and/or the packaging means are capable of being handled by means of the at least one handling device.

17. The system in accordance with claim 1,
wherein the system comprises a supply device for storing a plurality of containers.

18. The system in accordance with claim 1,
wherein the system comprises a preparation device, which is spatially separate from the apparatus and comprises a working space, wherein a removal opening that is capable of being closed and opened via a closing element is formed in a wall of the preparation device, wherein the container is dockable to the preparation device, and wherein the at least one production element is transferable from the working space into the interior when the closing elements are open.

19. The system in accordance with claim 1,
wherein the system comprises a plurality of reading units, which are arranged at different positions of the apparatus.

20. The system in accordance with claim 1,
wherein the at least one reading unit is stationary, and/or wherein at least one movable reading unit is provided.

21. The system in accordance with claim 1,
wherein a reading unit is arranged or fixed at at least one of the following positions:
outside the apparatus, wherein the reading unit is user-guided;
in the processing chamber, wherein the reading unit is user-guided;
on or to the at least one processing station;
on or to the at least one handling device in the processing chamber;
at the insertion opening, wherein the identification element of a container docked to the apparatus or to be docked to the apparatus is readable via the reading unit;
on or to the outside of the apparatus,
on or to a receiving element in the processing chamber for receiving a used or discarded production element;
in the working space of the preparation device;
on or to a handling device in the working space of the preparation device of the system, which is spatially separate from the apparatus and comprises a working space, wherein a removal opening that is capable of being closed and opened via a closing element is formed in a wall of the preparation device, wherein the container is dockable to the preparation device,
wherein the identification element of a container docked to the preparation device or to be docked to the preparation device is readable via the reading unit;
at a removal opening of a preparation device of the system, which is spatially separate from the apparatus and comprises a working space, wherein a removal opening that is capable of being closed and opened via a closing element is formed in a wall of the preparation device, wherein the container is dockable to the preparation device, wherein the identification element of a container docked to the preparation device or to be docked to the preparation device is readable via the reading unit.

22. The system in accordance with claim 1,
wherein the state information comprises or relates to at least one of the following:
an identification number of the production element;
a part number of the production element;
a type of the production element;
a batch of the production element;
information relating to the content of the production element in the case of a container as a production element;
information relating to the position of a further production element in the case of a container that receives the further production element;
information relating to the position of a further production element in the case of a receiving body that receives the further production element;
information relating to the geometry of the production element;
information relating to the installation of the production element in the apparatus regarding a target position in the apparatus;
information relating to a control of the apparatus using the production element in the apparatus;
information relating to a processing of the packaging means by the apparatus using the production element in the apparatus;
information indicating whether the production element has been used or has not yet been used;
information indicating whether the production element, in the case of a container, has been previously opened or not;
information indicating whether the production element is contaminated or not;
information as to whether the production element has been cleaned or sterilized or not;
a timestamp.

23. The system in accordance with claim 1,
wherein the apparatus comprises a control device, which is directly or indirectly coupled to the at least one reading unit, and wherein the apparatus is controllable depending on the determined state information.

24. The system in accordance with claim 23,
wherein the data processing device comprises or forms the control device, or wherein the data processing device is coupled to the control device.

25. The system in accordance with claim 23,
wherein at least one processing station is controllable by the control device.

26. The system in accordance with claim 23,
wherein at least one handling device, which is arranged in the processing chamber, for the production element and/or the packaging means is controllable by the control device.

27. The system in accordance with claim 1,
wherein at least one indicator unit for the user for providing at least one indication, comprising a controllable image display is controllable by the control device.

28. The system in accordance with claim 27,
wherein at least one of the following is providable on the at least one indicator unit, a production plan relating to at least one of a processing of the packaging means and a planned use of the at least one production element in the apparatus being provided:
a confirmation in the event that a production element identified on the basis of the state information is a correct production element, in accordance with the production plan;
an error indication in the event that a container that does not correspond to the production plan is docked or intended to be docked to the apparatus;
an error indication in the event that a container that assumes a state deviating from the production plan is docked or intended to be docked to the apparatus;
an error indication in the event that a container that does not contain at least one production element specified in the production plan or does not contain it in a specified state is docked or intended to be docked to the apparatus;
an error indication in the event that the container is docked or intended to be docked to the apparatus at an insertion opening deviating from an insertion opening specified in the production plan;
a confirmation if a container that is in accordance with the production plan is docked or intended to be docked to the apparatus.

29. The system in accordance with claim 1,
wherein the system comprises at least one self-propelled and self-steering transport device for receiving at least one container, wherein the container is capable of being autonomously docked to and decoupled from the apparatus via the transport device.

30. The system in accordance with claim 29,
wherein the at least one transport device comprises at least one reading unit, or wherein such a unit is arranged on the transport device.

31. A method for monitoring the system in accordance with claim 1,
wherein state information of at least one identification element is read by means of the at least one reading unit.
